(19) United States
(12) Patent Application Publication
(10) Pub. No.: US 2026/0097232 A1
(43) Pub. Date: Apr. 9, 2026

(54) THERAPEUTIC DEVICE FOR INFLAMMATORY, PAINFUL PATHOLOGY AND A NEURO-MUSCULAR AND POSTURAL REMODULATION

(71) Applicant: Fabio FONTANA, Castelfranco Veneto (TV) (IT)

(72) Inventor: Fabio FONTANA, Castelfranco Veneto (TV) (IT)

(21) Appl. No.: 19/114,158

(22) PCT Filed: Sep. 22, 2023
(86) PCT No.: PCT/IB2023/059402

§ 371 Date: Mar. 21, 2025

(30) Foreign Application Priority Data
Sep. 23, 2022 (IT) 102022000019506

(51) Int. Cl. A61N 5/06

(52) U.S. Cl. CPC A61N 5/0622; A61N 2005/0631; A61N 2005/0632; A61N 2005/0645; A61N 2005/0659; A61N 2005/0662

(57) ABSTRACT

Therapeutic device (1) for inflammatory, painful pathology and a neuro-muscular and postural remodulation of a person, which device (1) comprises at least one support laminar element (2), which laminar element (2) is made of material that is transparent to reference wavelengths, the reference wavelengths being comprised between 400 and 990 nm, a first side of the laminar element (2) being adapted to be associated in contact with the skin of said person, and on the second side of the laminar element (2), opposite to said first side, there being placed, or incorporated or diffused in the laminar element itself, at least one nanocrystal or a mixture of nanocrystals (3), which nanocrystal or mixture of nanocrystals (3) is able to emit photons in the reference wavelength when stressed by at least one infrared, light or ultraviolet electromagnetic radiation, which device comprises photoluminescent material (4).

The present invention relates to a therapeutic device for inflammatory, painful pathology and neuro-muscular and postural remodulation.

Innovative technologies and techniques are currently known, whose purpose is to integrate with the traditional and modern therapeutic treatments on the human body by increasing their effectiveness, in particular aiming at the treatment of inflammatory and painful pathologies or movement disorders and with the purpose of a postural and proprioceptive improvement.

Such a technology is disclosed by document WO 2021/084424 A1, which describes a therapeutic device capable of transmitting photons consistent with the human body. This therapeutic device constantly stimulates the neuralgic points that are in imbalance, without releasing any chemical substance, increases proprioception, and therefore constitutes an innovative technology for health and well-being, based on modern knowledge of biophysics applied to health and well-being.

This therapeutic device aims to improve the movement of the body and help the recovery of the joint functions, reduce any pain due to incorrect postural patterns, correct such incorrect postural patterns, thus favouring an energy recovery, which the body can use for an increase in the overall well-being.

Such a therapeutic device has the shape of a small foil element which is applied to the skin with the help of a patch tape. This can allow a postural balance to be maintained in the long term, integrating with the effects of the treatments under study.

Such a therapeutic device comprises at least one support laminar element and at least one nanocrystal. The laminar element is made of material that is transparent to reference wavelengths comprised between 400 and 990 nm. A first side of the laminar element is adapted to be associated in contact with the skin of the person, and on the second side of the laminar element, opposite to said first side, there is placed, or incorporated or diffused in the laminar element itself, at least one nanocrystal or a mixture of nanocrystals, which nanocrystal or mixture of nanocrystals is able to emit photons in the reference wavelength when stressed by at least one infrared, light or ultraviolet incident electromagnetic radiation.

However, the time of action of the device is strictly dependent on the stress of the infrared, light or ultraviolet electromagnetic radiation: when such stress is interrupted, the emission of photons by the nanocrystal or the mixture of nanocrystals is exhausted in the short term.

Document EP2383017A1 discloses a phototherapy patch adapted to relieve nociceptive pain of a part of the body by illuminating said part of the body. The patch is adapted to conform to the part of the body and comprises an active light source configured to emit light with a wavelength in the range from 430 nm to 475 nm, to illuminate the part of the body on which the patch is applied.

There is therefore a need not met by the state of the art for a device that significantly prolongs its time of action and reduces its dependence on an external electromagnetic stress, for greater autonomy.

The present invention aims to overcome this limit of the state of the art with a support device as described at the beginning, which further comprises photoluminescent material capable of absorbing photons, under the effect of said incident electromagnetic radiation, and of re-emitting them even after the stress of said incident electromagnetic radiation has ceased.

The photoluminescent material therefore absorbs photons, under the effect of said incident electromagnetic radiation, and then re-emits them, and this allows the action of the therapeutic device to be prolonged even after the external electromagnetic stress has ceased: the photons absorbed by the photoluminescent material and coming from the incident electromagnetic radiation are subsequently re-emitted by the photoluminescent material itself; these photons constitute the electromagnetic stress to the nanocrystal or to the mixture of nanocrystals; the nanocrystal, thus stressed, therefore emits photons in the reference wavelength even when the external electromagnetic radiation has ceased.

In a preferred embodiment, the device is passive, i.e. it is devoid of light sources.

In an embodiment example, a layer of said photoluminescent material is provided.

This allows to easily assemble the device starting from independent layers, each with its own specific functionality.

Alternatively or in combination it is possible to provide the photoluminescent material mixed or incorporated in the laminar element and/or in the mixture of nanocrystals.

According to a further embodiment, said nanocrystal or said mixture of nanocrystals forms an active layer and the layer of photoluminescent material is interposed between the laminar element and said active layer.

Keeping the photoluminescent material in direct contact with the active layer comprising the nanocrystal or the mixture of nanocrystals allows to optimize the electromagnetic stress effect exerted by the layer of photoluminescent material.

In one embodiment, the layer of photoluminescent material has a thickness comprised between 50 and 100 micrometres. In particular, the layer of photoluminescent material has a thickness comprised between 75 and 85 micrometres.

According to one embodiment the photoluminescent material comprises powdered fluorescent pigments diluted in ink.

In an executive example the fluorescent pigments are diluted in ink in a percentage comprised between 20% and 80%.

According to a particularly advantageous embodiment, the photoluminescent material comprises strontium aluminate.

This material has proved particularly advantageous because it allows to have a lasting photoluminescent action and does not have disadvantageous effects such as unpleasant odours or adverse reactions with the further elements of the device, for example the layer retention glue.

These and other features and advantages of the present invention will become clearer from the following disclosure of some embodiment examples illustrated in the accompanying drawings in which:

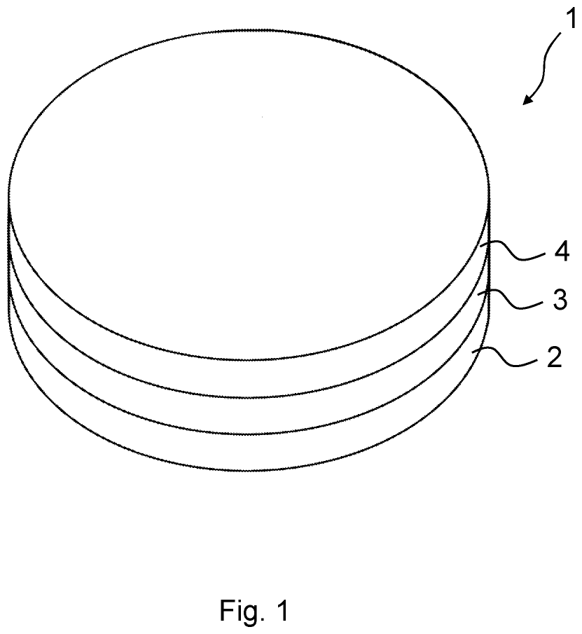

FIG. 1 illustrates a schematic view of a first embodiment of the therapeutic device;

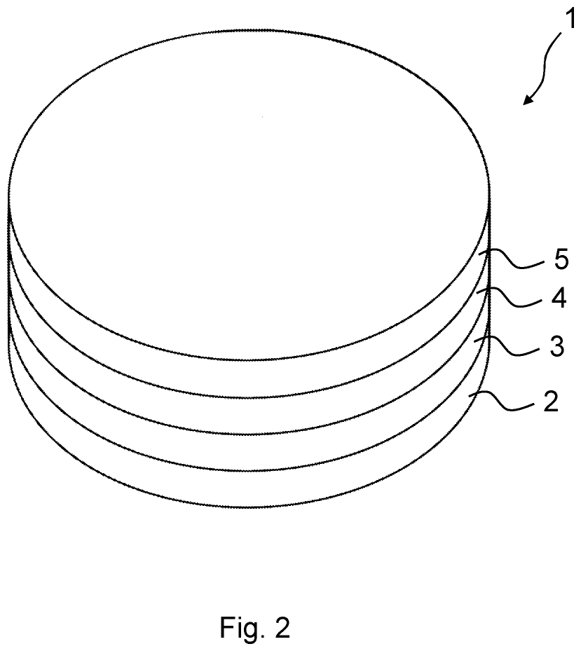

FIG. 2 illustrates a schematic view of a second embodiment of the therapeutic device.

Embodiments of the therapeutic device 1 for inflammatory, painful pathology and a neuro-muscular and postural remodulation of a person according to the present invention are illustrated in the figures. The figures are for illustrative purposes only and do not show the device to scale.

As illustrated in FIG. 1, the device 1 comprises a support laminar element 2 made of material that is transparent to reference wavelengths comprised between 400 and 990 nm. A first side of the laminar element 2 is adapted to be associated in contact with the skin of the person.

The laminar element 2 consists of a material transparent to at least one infrared, light, ultraviolet electromagnetic radiation. This material is polymeric and able to associate with the person's epidermis, being impermeable and inert with respect to the sweat emitted by the skin. The material is flexible and able to follow the movements and/or the deformations of the person's epidermis without detaching from it. Preferably the foil element 2 has a thickness comprised between 0.05 mm and 2 mm, and even more preferably comprised between 0.1 and 1 mm, being able to better adapt to the deformations of the epidermis.

On the second side of the laminar element 2, opposite the first side resting on the epidermis, there is placed at least one nanocrystal or a mixture of nanocrystals 3. The nanocrystal or the mixture of nanocrystals are able to emit photons in the reference wavelength when stressed by an infrared, light or ultraviolet electromagnetic radiation.

The nanocrystal or the mixture of nanocrystals may be incorporated or diffused in the laminar element 2. In this case, said at least one nanocrystal or said mixture of nanocrystals 3 are arranged on the support in a distributed manner occupying most of the surface of the side on which it is arranged, like a varnish, using the whole surface for a high transmission efficiency of said radiation, without being hindered by an overlap of the nanocrystals, with a thickness comprised between 0.001 and 1 mm.

Said at least one nanocrystal or said mixture of nanocrystals 3 can be arranged on the laminar element 2 in discrete zones, concentrating the flow of the radiation in delimited zones, in order to be significantly intense and be received through said laminar element 2, with thicknesses comprised between 0.005 and 1 mm.

Preferably a mixture of nanocrystals is placed on the laminar element 2 or incorporated or diffused in the laminar element 2 itself, with a concentration from 1 mg/cm² to 6 g/cm² and able to emit photons in the reference wavelength.

Alternatively, or in combination, said at least one nanocrystal or said mixture of nanocrystals may be comprised in an independent layer to form an active layer 3.

The nanocrystal or the mixture of nanocrystals preferably comprises one or more of the following quantum dots:
Graphene quantum dots code 900708 or quantum dots with a fluorescence indicatively corresponding to: λex 350 nm; λem 445 nm, FWHM 65 nm, quantum yield>65%;
Blue luminescent graphene quantum dots, code 900726 or quantum dots with a fluorescence indicatively corresponding to: λex 350 nm; λem 445 nm+10 nm, FWHM 75 nm, quantum yield>20%
Cyan luminescent graphene quantum dots, code 900707 or quantum dots with a fluorescence indicatively corresponding to: λem 475-495 nm, FWHM 70 nm, quantum yield>17%
Aqua green luminescent graphene quantum dots, code 900712 or quantum dots with a fluorescence indicatively corresponding to: λex 485 nm; λem 530 nm+10 nm, FWHM 80 nm, quantum yield>17%;
Perovskite quantum dots coated with oleic acid and oleylamine, code 900747 or quantum dots with a fluorescence approximately corresponding to: λem 480 nm;
COOH functionalised core-type CdTe quantum dots, code 777978 or quantum dots with a fluorescence indicatively corresponding to: λem 710 nm, quantum yield>15%;
CdS/ZnS functionalised oleic acid core-shell type quantum dots, code 900286 or quantum dots with a fluorescence indicatively corresponding to: λmax 385 nm λem 400 nm±10 nm, quantum yield>50%;
CdS/ZnS functionalised oleic acid core-shell type quantum dots, code 900283 or quantum dots with a fluorescence approximately corresponding to: λmax 405 nm λem 425 nm±10 nm;

The nanocrystals or the mixture of nanocrystals allows to reach and produce the same wavelengths as ULLLT (ultra-low-level laser therapy) but with ultra-weak intensity.

The device further comprises photoluminescent material 4, preferably provided in a photoluminescent layer 4 separated from the other components of the device. Alternatively or in combination it is possible to provide the photoluminescent material 4 mixed or incorporated in the laminar element and/or in the mixture of nanocrystals.

Preferably the layer of photoluminescent material 4 is interposed between the laminar element 2 and said active layer 3 and has a thickness comprised between 50 and 100 micrometres, in particular comprised between 75 and 85 micrometres.

Preferably the photoluminescent material 4 comprises powdered fluorescent pigments diluted in ink, in particular in a percentage comprised between 20% and 80%.

In a preferred embodiment, the photoluminescent material 4 comprises strontium aluminate. This is particularly advantageous because a photoluminescent layer comprising strontium aluminate in the thicknesses indicated above absorbs photons, in particular UV, up to a maximum state of recharge in a few minutes and releases photoluminescence with significant duration, even up to twenty-four hours.

It is possible alternatively or in combination to use other rare earths or other suitable photoluminescent materials, such as for example barium titanate, zinc sulphate, or the like.

The photoluminescent layer 4 can be provided in various emission colours such as for example yellow-green, green, blue-green, purple, orange.

FIG. 2 illustrates a second embodiment, in which a second laminar element 5 is provided for protecting and confining the photoluminescent layer 3. The second laminar element 5 consists of a material transparent either to infrared radiation or to light radiation in the visible or ultraviolet spectrum, so that the radiation coming from outside and passing through said second laminar element 5 can recharge the photoluminescent layer 4 and excite the aforementioned mixture of nanocrystals 3 to emit the frequencies of interest.

The support laminar element 2 and the second protection and confinement laminar element 5 comprise between them and confine said nanocrystal or said mixture of nanocrystals 3, protecting them from external agents or from mechanical stresses that could lead to damage.

The second laminar element 5 has an extension comparable to the extension of the support laminar element 2, it is associated leaning against it with the surface of its side turned toward the laminar element 2, retaining and tight-sealingly confining the nanocrystal or the mixture of nanocrystals 3 and the photoluminescent layer 4.

The second laminar element 5 is preferably constituted by a protective transparent ink or a film of polymeric material, managing to keep the overall thickness of the device reduced and making it better wearable.

The mixture of nanocrystals 3 can be diluted within an ink to be able to be printed on the laminar element 2 and/or on the second protective laminar element 5, directly on the mutually facing sides. It is also possible to dilute the pigments of the photoluminescent material 3 in the same ink.

The wearable therapeutic device carries out a greater activity of the nanocrystal or of the mixture of nanocrystals 4 with a mixture of nanocrystals from 95 to 20%, depending on the type of nanocrystals used and the frequency of interest of the therapeutic radiation, managing to calibrate, redefine and dose the quantity of photons emitted by the device according to the needs of the patient and the stimulus it is wished to be obtained.

The ink is preferably a transparent ink, allowing to the characteristics only of the glue and of the ink to still be exerted in the dispersion medium, and that all the outgoing photon emissions and the incoming radiations can reach the nanocrystal or the nanocrystal mixture 3 with minimal attenuation.

The total thickness of the device is minimal in order to maintain a high elasticity and to be able to adapt to the surface stresses of the dermis without cracking, breaking or tearing.

The mutual union between dermis and laminar element 2 and/or second laminar element 5 can be obtained by means of a flexible double-adhesive material.

The fact that the device can be mostly transparent, and by associating with an adhesive effect to the skin, placed near primary and secondary endings, tendons, muscles, dermatomers, nerve endings, to favour a neuro-muscular and postural remodulation, means that it is possible to check the skin for any redness.

1. Therapeutic device (1) for inflammatory, painful pathology and a neuro-muscular and postural remodulation of a person, which device (1) comprises at least one support laminar element (2), which laminar element (2) is made of material that is transparent to reference wavelengths, the reference wavelengths being comprised between 400 and 990 nm, a first side of the laminar element (2) being adapted to be associated in contact with the skin of said person, and on the second side of the laminar element (2), opposite to said first side, there being placed, or incorporated or diffused in the laminar element itself, at least one nanocrystal or a mixture of nanocrystals (3), which nanocrystal or mixture of nanocrystals is able to emit photons in the reference wavelength when stressed by at least one infrared, light or ultraviolet incident electromagnetic radiation,
characterized in that
it comprises photoluminescent material (4) capable of absorbing photons, under the effect of said incident electromagnetic radiation, and of re-emitting them even after the stress of said incident electromagnetic radiation has ceased.

2. Device according to claim 1, wherein the device is passive, i.e. it is devoid of light sources.

3. Device according to claim 1, wherein a layer of said photoluminescent material (4) is provided.

4. Device according to claim 3, wherein said nanocrystal or said mixture of nanocrystals forms an active layer (3) placed in contact with the laminar element (2) and the layer of photoluminescent material (4) is placed in contact with said active layer (3) on the side thereof opposite with respect to the side in contact with the laminar element (2).

5. Device according to claim 1, wherein the layer of photoluminescent material (4) has a thickness comprised between 50 and 100 micrometres.

6. Device according to claim 1, wherein the photoluminescent material (4) comprises powdered fluorescent pigments diluted in ink.

7. Device according to claim 4, wherein the fluorescent pigments are diluted in ink in a percentage comprised between 20% and 80%.

8. Device according to claim 1, wherein the photoluminescent material (4) comprises strontium aluminate.
